(19) United States
(12) Patent Application Publication
(10) Pub. No.: US 2022/0338879 A1
(43) Pub. Date: Oct. 27, 2022

(54) NON-PNEUMATIC DIGITAL TOURNIQUET

(71) Applicant: Mar-Med Inc., Grand Rapids, MI (US)

(72) Inventor: Jerry Paul Marogil, Grand Rapids, MI (US)

(21) Appl. No.: 17/659,726

(22) Filed: Apr. 19, 2022

Related U.S. Application Data
(60) Provisional application No. 63/177,983, filed on Apr. 22, 2021.

(51) Int. Cl. A61B 17/132

(52) U.S. Cl. CPC A61B 17/1325; A61B 2017/00862

(57) ABSTRACT

A non-pneumatic tourniquet includes a tourniquet body including a strip of resiliently elastic material. The tourniquet body includes a tourniquet section and a pair of handle sections on opposite ends of the tourniquet section. The tourniquet section defines at least two holes sized and shaped to receive a body part of a subject. The tourniquet section is configured to restrict blood flow to the body part when the body part is received in one of the holes in the tourniquet section.

### CROSS-REFERENCE TO RELATED APPLICATION

This application claims priority to U.S. Provisional Application No. 63/177,983, filed Apr. 22, 2021, the entire content of which is hereby incorporated by reference.

### FIELD

The present disclosure generally relates to non-pneumatic tourniquets, and more particularly to non-pneumatic digital tourniquets.

### BACKGROUND

A digital tourniquet is a constricting or compressing device, used to control venous and arterial circulation to a digit for a period of time. Digital tourniquets are commonly used in emergency medicine and orthopedic surgical procedures and enable clinicians to work in a bloodless operative field by preventing blood flow to a digit. With better visibility, tourniquets allow procedures to be performed with improved precision, safety and speed.

Non-pneumatic digital tourniquets are generally made with an elastic material that provides compression by stretching and exerting pressure or through manually tightening. They are simple, low cost, and easily disposable, thus appropriate for digit procedures of short duration and limited seriousness. Depending on the design non-pneumatic tourniquets can expel venous blood and occlude arterial circulation in one quick step. And they can be designed to be low profile in order to allow the surgeon greater access to the surgical site.

Some non-pneumatic digital tourniquets come in multiple sizes in order to accommodate the various sizes of digits, from small toes and pediatric digits to large toes and full adult fingers. Other non-pneumatic digital tourniquets are elastic discs with a single hole that conforms to a digit once passed through. This style is considered more universal in size because the disc conforms to the size of the digit, with greater surface area on larger digits dissipating the force (pressure=force/surface area). However, in practice even with the disc style devices it is difficult to effectively cover a wide range of digit sizes.

Therefore, there exists a need for a non-pneumatic digital tourniquet which is reasonably priced, easy to use and universally able to provide a bloodless environment.

### SUMMARY

A non-pneumatic tourniquet apparatus for improving an operating field on a digit of a patient comprising a flexible membrane and multiple membrane openings. The multiple openings in the membrane can vary in size and shape and the membrane itself can vary in size, shape and thickness in order to provide for the appropriate level of exsanguination and compression on any digit size.

In one aspect, a non-pneumatic tourniquet generally comprises a tourniquet body comprising a strip of resiliently elastic material. The tourniquet body includes a tourniquet section and a pair of handle sections on opposite ends of the tourniquet section. The tourniquet section defines at least two holes sized and shaped to receive a body part of a subject. The tourniquet section is configured to restrict blood flow to the body part when the body part is received in one of the holes in the tourniquet section.

In another aspect, a method of limiting blood flow to a body part of a subject generally comprises stretching a tourniquet section of a non-pneumatic tourniquet to position the tourniquet section over a distal end portion of the body part. The tourniquet section defines at least two holes sized and shaped to receive the body part. The method further comprising applying a force to a pair of handle sections of the tourniquet attached to opposite ends of the tourniquet section to slide the tourniquet proximally along the body part as the tourniquet section constricts around the body part to limit blood flow to the body part.

### BRIEF DESCRIPTION OF THE DRAWINGS

FIG. 1 is a perspective of a tourniquet of the present disclosure;

FIG. 2 is a top plan view of the tourniquet;

FIG. 3 is a side view of the tourniquet; and

FIG. 4 is a section of the tourniquet taken through line 4-4 in FIG. 2.

Corresponding reference characters indicate corresponding parts throughout the drawings.

### DESCRIPTION OF THE PREFERRED EMBODIMENTS

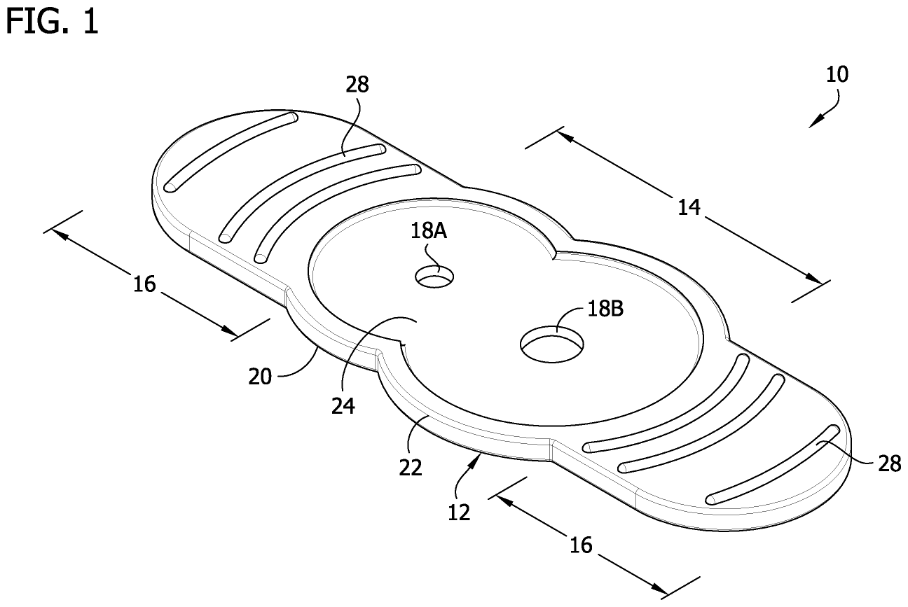

Referring to FIGS. 1-3, a non-pneumatic tourniquet is generally indicated at reference number 10. The tourniquet 10 comprises a tourniquet body 12 including a tourniquet section 14, and a pair of handle sections 16 on opposite ends of the tourniquet section. In the illustrated embodiment, the tourniquet body 12 comprises a single unitary structure. However, the tourniquet body 12 may comprise multiple structures suitably attached together without departing from the scope of the disclosure. The tourniquet section 14 of the tourniquet body 12 defines at least two holes 18A, 18B sized and shaped to receive a body part of a subject (e.g., a human or animal surgical patient) and configured to restrict blood flow to the body part when it is received in one of the holes. The handle sections 16 are configured for grasping the tourniquet body 12 and pulling the tourniquet 10 onto the body part of the subject. In one embodiment, the tourniquet 10, and more particularly the holes 18A, 18B are sized and shaped to receive a digit (i.e., fingers and toes) of a subject and configured to operatively restrict blood flow to the subject's digit. The tourniquet section 14 can be sized and arranged to constrict around a relatively short axial segment of the body part. Other embodiments of tourniquets may be configured to operatively engage and restrict blood flow at other body parts such as arms and legs.

The tourniquet 10 may suitably be made of inexpensive materials that can be manufactured, packaged in sterile packaging, and delivered to a treatment site, such as an operating room. As will be apparent, the tourniquet 10 may have a relatively narrow profile to minimize the size of the segment of the body part that is covered by the tourniquet in use, and thereby maximize the size of the surgical field. As explained below, the handle sections 16 allow a user to pull the tourniquet 10 over the body part of the subject using a two-handed grip. And as the tourniquet 10 is pulled over the body part, the tourniquet section 14 slides along the body part and exsanguinates the body part. Once the tourniquet 10 is properly positioned, it constricts tightly around a segment of the body part to restrict blood flow to the portions of the body part extending distally from the constricted segment. Suitably, the tourniquet 10 is configured to impart sufficient pressure on the body part to overcome venous blood pressure as it is pulled onto the body part, thereby exsanguinating the body part; likewise, the tourniquet is configured to impart sufficient pressure on the body part to overcome arterial blood pressure at least when it is properly positioned on the body part, thereby restricting new blood flow to the surgical site after the tourniquet is placed.

The tourniquet body 12 comprises an elongate strip of resiliently elastic material. In one or more embodiments, the tourniquet body 12 is flexible (e.g., bendable or extendable) about/along 3 axes (e.g., x-y-z axes). Thus, the tourniquet body 12 is easily manipulated to place the tourniquet 10 over the subject's body part. The illustrated tourniquet body 12 has a generally racetrack shape, including a multi-lobed center tourniquet section 14. However, tourniquet bodies of other shapes may also be used in other embodiments. The tourniquet body 12 may suitably comprise a silicone. However, other resiliently elastic materials may also be used without departing from the scope of the disclosure.

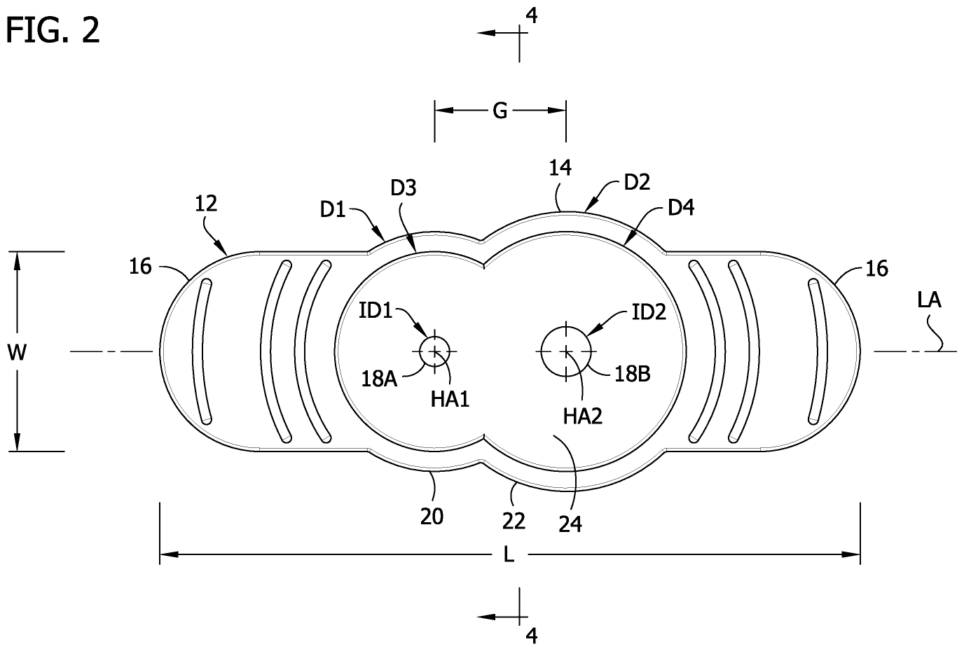

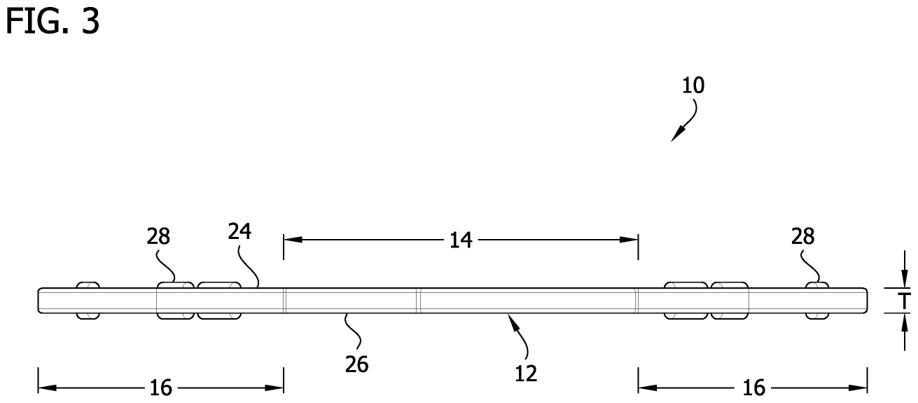

Referring to FIGS. 2 and 3, the tourniquet body 12 has a length L extending along a longitudinal axis LA between opposite free ends of the tourniquet body. The length L of the tourniquet body 12 may suitably be in a range of from about 50 millimeters (2 inches) to about 90 millimeters (3.5 inches). A width W of the tourniquet body 12 extends between opposite longitudinally extending sides of the tourniquet body. In one embodiment, the width W of the tourniquet body 12 may suitably be in a range of from about 10 millimeters (0.4 inches) to about 30 millimeters (1.2 inches). In the illustrated embodiment, the width W is generally a maximum width of the handle sections 16 of the tourniquet body 12. However, the tourniquet section 14 of the tourniquet body 12 has a bulbous or lobed shape comprising a first bulbous section 20 and a second bulbous section 22. Each bulbous section 20, 22 defines a portion of a circle having a diameter that is larger than the width W of the handle sections 16. Thus, the tourniquet section 14 has widths that are greater than the widths of the handle sections 16. However, the width of the tourniquet section 14 could be the same or less than the width of the handle sections 16 without departing from the scope of the disclosure. In the illustrated embodiment, the second bulbous section 22 has a diameter D2 that is larger than a diameter D1 of the first bulbous section 20. In one or more embodiments, the diameter D1 may be in a range of from about 20 millimeters (0.79 inches) to about 30 millimeters (1.18 inches). In one or more embodiments, the diameter D2 may be in a range of from about 25 millimeters (0.99 inches) to about 35 millimeters (1.38 inches). Still other dimensions for the tourniquet section 14 are envisioned.

The holes 18A, 18B in the tourniquet section 14 are sized and arranged such that the tourniquet section provides a sufficient resiliently compressive force when stretched around the body part (e.g., digit) to restrict blood flow. Referring to FIG. 2, a first hole 18A defines a non-stretched inner diameter ID1, and a second hole 18B defines a non-stretched inner diameter ID2. In the illustrated embodiment, the non-stretched ID1 of the first hole 18A is smaller than the non-stretched ID2 of the second hole 18B. Thus, the second hole 18B circumscribes a larger area configured to receive a body part having a larger cross-sectional dimension than a body part the first hole 18A is configured to receive. The non-stretched inner diameters ID1, ID2 of both holes 18A, 18B may suitably be less than the effective outer diameter of a body part so that the tourniquet section 14 around the holes must be stretched circumferentially to be pulled onto the body part. The required stretching may be accomplished by radially expanding the holes 18A, 18B as the tourniquet 10 is being placed on the body part B. After being stretched, the tourniquet section 14 resiliently returns toward its non-stretched configuration and constricts around the body part to restrict blood flow. In one or more embodiments, the non-stretched inner diameter ID1 of the first hole 18A may be in a range of from about 2 millimeters (0.08 inches) to about 4 millimeters (0.16 inches). In one or more embodiments, the non-stretched inner diameter ID2 of the second hole 18B may be in a range of from about 4 millimeters (0.16 inches) to about 6 millimeters (0.24 inches). Other sizes of the holes 18A, 18B are also envisioned.

The holes 18A, 18B have central axes HA1, HA2, respectively, extending through the holes. The holes 18A, 18B are centered on the tourniquet body 12 such that the central axes HA1, HA2 are disposed on the longitudinal axis LA of the tourniquet body. The holes 18A, 18B are suitably spaced apart from each other to allow for each hole to be used independently without being interfered with by the other hole. In one or more embodiments, the first hole 18A is spaced from the second hole 18B by a gap G extending between the axes HA1, HA2 of the holes 18A, 18b in a range between about 10 millimeters (0.39 inches) and about 15 millimeters (0.59 inches). Still other dimensions are envisioned without departing from the scope of the disclosure. Further, while the holes 18A, 18B are shown as circular openings, the holes could have other configurations. For example, the holes 18A, 18B may comprise slits or elongate/oblong openings. Still other shapes are envisioned.

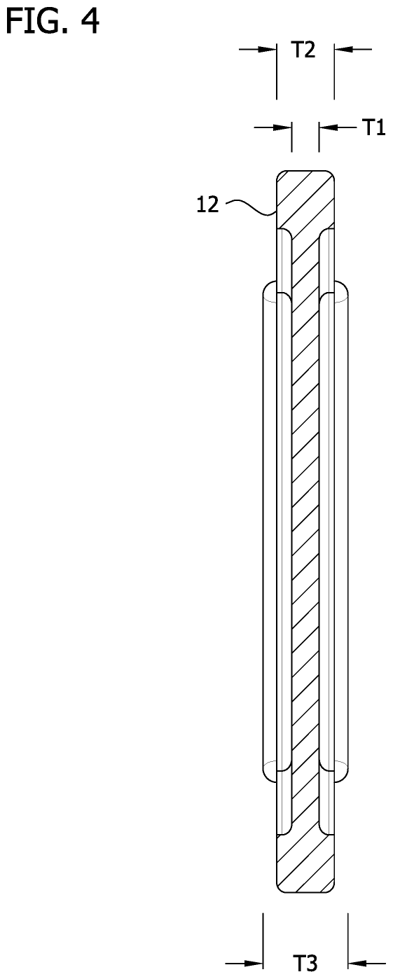

A thickness T (FIG. 3) of the tourniquet body 12 extends between the upper and lower surfaces 24, 26 of the tourniquet body. The thickness T of the tourniquet body 12 may vary across the tourniquet body. In the illustrated embodiment, a thickness T1 (FIG. 4) of the tourniquet body 12 in the tourniquet section 14 is less than a thickness T2 of the tourniquet body at the handle sections 16. The reduced thickness T1 of the tourniquet body 12 at the tourniquet section 14 provides a more flexible section of the tourniquet body configuring the tourniquet to receive the subject's body part through the holes 18A, 18B in the tourniquet section and conform the shape of the hole to the body part. In the illustrated embodiment, the reduced thickness T1 of the tourniquet body 12 is contained generally within an interior portion of the bulbous sections 20, 22 of the tourniquet section 14, and the reduced thickness T1 of the tourniquet body 12 defines the holes 18A, 18B in the tourniquet section. However, the thickness of the tourniquet body 12 at the tourniquet section 14 is still configured to provide sufficient elastic compression strength to restrict blood flow when the tourniquet section resiliently constricts around the body part. In one or more embodiments, a diameter D3 (FIG. 2) of the reduced thickness portion in the first bulbous section 20 may be in a range from about 18 millimeters (0.71 inches) to about 22 millimeters (0.87 inches). In one or more embodiments, a diameter D4 of the reduced thickness portion in the second bulbous section 22 may be in a range from about 22 millimeters (0.87 inches) to about 26 millimeters (1.02 inches). In one embodiment, the reduced thickness portion of the tourniquet section 14 may be broadly considered a flexible membrane.

Referring to FIGS. 1 and 3, ridges 28 may be formed on the upper and lower surfaces 24, 26 at the handle sections 16 to provide grip surfaces for grasping the tourniquet 10. In the illustrated embodiment, the ridges 28 have a generally arc shape extending across the width W of the tourniquet body 12. A thickness T3 (FIG. 4) of the tourniquet body 12 extending through the ridges 28 is greater than the thickness T2 of the tourniquet body. This is a result of the ridges 28 extending outward from the upper and lower surfaces 24, 26 of the tourniquet body 12. In one or more embodiments, the thickness T1 of the tourniquet body 12 may be in a range of from about 0.5 millimeters (0.02 inches) to about 1.5 millimeters (0.06 inches). In one or more embodiments, the thickness T2 of the tourniquet body 12 may be in a range of from about 1.5 millimeters (0.06 inches) to about 2.5 millimeters (0.1 inches). In one or more embodiments, the thickness T3 of the tourniquet body 12 may be in a range of from about 2.5 millimeters (0.1 inches) to about 3.5 millimeters (0.14 inches). Still other dimensions are envisioned without departing from the scope of the disclosure.

An exemplary method of using the tourniquet 10 will now be briefly described. As discussed above, the tourniquet 10 may be configured as a one-time use device. In one embodiment, a user receives the tourniquet 10 in sterile packaging and must remove the packaging before positioning the tourniquet. Initially, the user selects between the first hole 18A and a second hole 18B of the tourniquet section 14 for receiving a body part therein. The selection may be based on the size and/or shape (e.g., cross-sectional dimension) of the body part. For example, a body part having a size at or below a predetermined amount may be selected to be received in the first hole 18A, and a body part having a size above a predetermined amount may be selected to be received in the second hole 18B. The user may then manipulate the tourniquet 10 and the body part to stretch the tourniquet section 14 of the tourniquet body 12 over the distal end portion of the body part (e.g., the digit of a subject may be contorted to fit inside one of the selected opening 18A, 18B). In general, the tourniquet 10 is positioned by grasping the handle sections 16, one with each hand, and sliding the tourniquet body 12 onto the body part along the hole axis HA1, HA2 of one of the holes 18A, 18B. Thus, to effectively pull or push the tourniquet 10 onto the body part, the pulling or pushing forces (generically, positioning forces) should be concentrated along the hole axis HA1, HA2 of the hole into which the body part is being received. During pulling/pushing, the tourniquet section 14 imparts a radially compressive force on the body part to exsanguinate the body part. When the tourniquet 10 reaches the desired positon, the tourniquet section 14 constricts around a segment of the body part to restrict blood flow.

Alternatively, the user may first select the second hole 18B and then manipulate the tourniquet 10 and the body part to stretch the second hole over the distal end portion of the body part. The user may then asses the fit of tourniquet 10 around the body part. If the fit is sufficient, such that blood flow is adequately restricted, then the user may leave the tourniquet in place. However, if it is determined that the fit does not adequately restrict blood flow, the user may remove the tourniquet 10 and reapply the tourniquet using the first hole 18A.

In one or more methods of using the tourniquet 10, a lubricant may be used to facilitate sliding the tourniquet along the body part. Suitably, the lubricant may comprise a surgical antiseptic material for reducing the likelihood of infection, sepsis, or putrefaction. Exemplary lubricant materials include Hibiclens®, sold by Mölnlycke Health Care AB of Gothenburg, Sweden and ChloraPrep® sold by Becton, Dickinson and Company of Franklin Lakes, N.J. Before or after the lubricant dries, or is otherwise wiped away, the user applies the tourniquet 10 to the body part as described above. In addition to (optionally) providing an antiseptic treatment of a surgical site prior to a surgical procedure, the lubricant provides a lubricated interface between the tourniquet body 12 and the body part, thereby reducing frictional resistance to sliding. The lubricant can also be iodine or blood. Also, the lubricant can be in the form of a coating that is applied to the product to allow it to slide easily.

In one or more embodiments, a tag (now shown) may extend through the holes 18A, 18B and be secured to the tourniquet body 12. The tag may provide a visual reminder to remove the tourniquet 10 once the surgical procedure is complete. The tag may also be grasped by the user to assist in removing the tourniquet 10 from the subject's body part after the surgical procedure.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A non-pneumatic tourniquet comprising:
a tourniquet body comprising a strip of resiliently elastic material, the tourniquet body including a tourniquet section and a pair of handle sections on opposite ends of the tourniquet section, the tourniquet section defining at least two holes sized and shaped to receive a body part of a subject, the tourniquet section being configured to restrict blood flow to the body part when the body part is received in one of the holes in the tourniquet section.

2. The non-pneumatic tourniquet set forth in claim 1, wherein the at least two holes comprise a first hole and a second hole, the second hole being larger than the first hole.

3. The non-pneumatic tourniquet set forth in claim 2, wherein the holes are circular.

4. The non-pneumatic tourniquet set forth in claim 3, wherein a non-stretched inner diameter of the first hole is between about 2 millimeters (0.08 inches) to about 4 millimeters (0.16 inches), and the non-stretched inner diameter of the second hole is between about 4 millimeters (0.16 inches) to about 6 millimeters (0.24 inches).

5. The non-pneumatic tourniquet set forth in claim 1, wherein the holes are centered about a longitudinal axis of the tourniquet body.

6. The non-pneumatic tourniquet set forth in claim 5, wherein the holes are spaced apart by a gap extending between centers of the holes in a range between about 10 millimeters (0.39 inches) and about 15 millimeters (0.59 inches).

7. The non-pneumatic tourniquet set forth in claim 1, wherein the tourniquet section has a first thickness and the pair of handle sections have a second thickness, the first thickness being less than the second thickness.

8. The non-pneumatic tourniquet set forth in claim 7, wherein the first thickness is in a range of from about 0.5 millimeters (0.02 inches) to about 1.5 millimeters (0.06 inches), and the second thickness is in a range of from about 1.5 millimeters (0.06 inches) to about 2.5 millimeters (0.1 inches).

9. The non-pneumatic tourniquet set forth in claim 1, wherein the tourniquet section is configured to receive a digit of the subject and restrict blood flow to the digit when the digit is received in one of the holes.

10. The non-pneumatic tourniquet set forth in claim 1, wherein the tourniquet section comprises first and second bulbous sections.

11. The non-pneumatic tourniquet set forth in claim 10, wherein the second bulbous section has a diameter that is larger than a diameter of the first bulbous section.

12. The non-pneumatic tourniquet set forth in claim 10, wherein the first bulbous section defines a first hole and the second bulbous section defines a second hole.

13. A method of limiting blood flow to a body part of a subject, the method comprising:
stretching a tourniquet section of a non-pneumatic tourniquet to position the tourniquet section over a distal end portion of the body part, the tourniquet section defining at least two holes sized and shaped to receive the body part; and
applying a force to a pair of handle sections of the tourniquet attached to opposite ends of the tourniquet section to slide the tourniquet proximally along the body part as the tourniquet section constricts around the body part to limit blood flow to the body part.

14. The method set forth in claim 13, further comprising selecting between a first hole and a second hole of the tourniquet section for receiving the body part.

15. The method set forth in claim 14, wherein the second hole is larger than the first hole.

16. The method set forth in claim 14, wherein the holes are circular.

17. The method set forth in claim 14, wherein the holes are centered about a longitudinal axis of the tourniquet.

18. The method set forth in claim 13, wherein the tourniquet section has a first thickness and the pair of handle sections have a second thickness, the first thickness being less than the second thickness.

19. The method set forth in claim 13, wherein the tourniquet section comprises first and second bulbous sections, the second bulbous section having a diameter that is larger than a diameter of the first bulbous section.

20. The method set forth in claim 13, further comprising stretching the tourniquet section around a digit of the subject.
